(19) United States
(12) Patent Application Publication
(10) Pub. No.: US 2023/0143825 A1
(43) Pub. Date: May 11, 2023

(54) A NANOPARTICLE FOR USE IN THE TREATMENT OF AN OCULAR DISEASE

(71) Applicant: UNIVERSITÄT REGENSBURG, REGENSBURG (DE)

(72) Inventors: MARILENA BOHLEY, REGENSBURG (DE); ACHIM GÖPFERICH, SINZING (DE)

(21) Appl. No.: 17/910,239

(22) PCT Filed: Mar. 11, 2021
(86) PCT No.: PCT/EP2021/056231

§ 371 Date: Sep. 8, 2022

(30) Foreign Application Priority Data
Mar. 11, 2020 (EP) 20162277.6

(51) Int. Cl. A61K 9/127; A61K 38/13; A61P 27/02

(52) U.S. Cl. CPC A61K 9/1271; A61K 38/13; A61P 27/02

(57) ABSTRACT

The present invention relates to a nanoparticle for use in the treatment of ocular diseases, in particular diseases of the retina (“retinopathies”) or of optic neuropathies, in particular glaucoma.

The present invention relates to a nanoparticle for use in the treatment of ocular diseases, in particular diseases of the retina (“retinopathies”) and optic neuropathies, in particular glaucoma.

Diseases of the retina or “retinal diseases” or “retinopathies” are diseases or disorders that affect the retina of a patient and typically result in or are associated with vision impairment in the patient. Retinal diseases may or may not include hereditary aspects as well as an involvement of a damage, remodelling or new formation of blood vessels that supply the retina. In the latter case, such retinal diseases involving a damage, remodelling or new formation of blood vessels supplying the retina are also sometimes referred to as “neovascular ocular diseases”.

Neovascular ocular diseases, including age-related macular degeneration (AMD), diabetic retinopathy (DR) and retinopathy of prematurity (ROP) are all among the leading causes of blindness, equally effecting adults and children globally [WHO, Vision 2020: The Right to Sight]. With an increasing number of people suffering from diabetes and with an aging population, a dramatic increase in the number of new cases is expected [Bourne, R. R. A. et al., Magnitude, temporal trends, and projections of the global prevalence of blindness and distance and near vision impairment: a systematic review and meta-analysis., The Lancet, 5(9), 2017].

Although, the localizations of neovascularization differ, the diseases share the same pathomechanism that is characterized by elevated levels of inflammatory mediators and enormous delocalization of blood vessels in the posterior segment of the eye, leading to massive damage of the retina and ultimately causing visual impairment and blindness. The main responsible factor for the initiation and development of choroidal and retinal neovascularization is the vascular endothelial growth factor (VEGF), that is primarily expressed by retinal pigment epithelium (RPE) cells. VEGF then promotes the proliferation and hyperpermeability of endothelial cells [O. Strauss, The Retinal Pigment Epithelium in Visual Function., Physiol. Rev., 85, 2005].

The recognition of VEGF as a key factor in the pathogenesis led to present standard therapy for the treatment of all neovascular ocular diseases, intravitreal anti-VEGF antibody injections. Even though this therapeutic concept has shown great success, there are numerous drawbacks and side effects that come along with a continuous anti-VEGF therapy. That is not surprising, since the abundant biological effects of VEGF are not limited to endothelial cells, where the effects are undesired, but affect various other cell types such as Muller cells, astrocytes, ganglion cells, photoreceptors and RPE cells. With VEGF being a survival factor for all these cell types, appropriate levels of VEGF are vital for ocular homeostasis and integrity [K. M. Ford, et al., Expression and role of VEGF in the adult retinal pigment epithelium., iOVS, 52, 2011].

Clinically, the rigorous suppression of omnipresent VEGF levels manifests itself in a decrease of choroid thickness and contributes to vitreoretinal fibrosis and geographic atrophy, which is accompanied by a local massive cell death of RPE cells and photoreceptors [Falavarjani K. G., Adverse events and complications associated with intravitreal injection of anti-VEGF agents: a review of literature., Eye, 27, 2013]. In addition, there have been reports of RPE tears after administration of VEGF neutralizing agents, suggesting that neutralization of VEGF may have adverse effects [B. Yeh, S. Ferrucci, Retinal pigment epithelium tears after bevacizumab injection. Optometry, 82, 2011]. Moreover, recent observations raise questions regarding the efficacy of these treatments beyond 2 years [Y. Tao, et al., Long-term follow-up after multiple intravitreal bevacizumab injections for exudative age-related macular degeneration. J. Ocular Pharmacol. Ther., 26, 2010]. A recent report described significant vision loss after 2 years of anti-VEGF treatment that appeared to be unassociated with the primary pathology, raising possibility of damage to the RPE and photoreceptors from “off-target” effects of VEGF neutralization [P. J. Rosenfeld et al., Characteristics of patients losing vision after 2 years of monthly dosing in the phase III ranibizumab clinical trials., Ophthalmology, 118, 2011].

Glaucoma represents one of the most common causes for blindness and currently affects more than 60 million people. Because of an increased life expectancy and ageing of populations, a dramatic increase of cases of glaucoma is expected. Clinically, glaucoma manifests itself as an optic neuropathy which leads from irreversible losses of vision to a complete blindness because of a persistent damaging of the optic nerve. Pathologically, glaucomas are optic neuropathies characterized by degeneration of retinal ganglion cells concomitant with changes in the optic nerve head. Although it is known that the retinal ganglion cells that concur in the optic nerve as well as their axons are damaged already at an early stage of the disease, and although the death of each cell in the optic nerve leads to a loss of vision, the etiology of the disease is not known or understood. Factors that contribute to a damaging are, inter alia, an increased internal pressure of the eye. Patients having glaucoma and an increased internal pressure of the eye are treated by means to reduce such internal pressure of the eye and to thereby slow progress of the disease. The standard therapy includes topically applied eye drops which are intended to reduce the internal pressure by promoting the efflux of the aqueous humor or by reducing the formation of such humor in the first place. Patient compliance is unfortunately, however, low in such a long term therapy which often times leads to failure of therapy. In view of the frequency of this chronical disease and the lack of effective therapies, there is an urgent need for means to intervene with the development and progression of the disease and which may help to protect and regenerate the optic nerve. The therapeutic system in accordance with the present invention allows, to transport encapsulated drugs, specifically to retinal pigment epithelial (RPE) cells, endothelial cells, and/or optic nerve cells, after systemic application and to successfully treat one or several of an inflammatory component, an immune-response component, an angiogenic component and a neurodegenerative component of a retinal disease or of an optic neuropathy.

According to a first aspect, the present invention relates to a nanoparticle comprising
a core comprising a drug that has one or several of the following activities: anti-inflammatory activity, immune-suppressive activity, anti-angiogenic activity, neuroprotective activity, gene therapeutic activity and regulatory activity on gene expression;
an amphiphilic shell surrounding said core, said amphiphilic shell comprising at least one phospholipid and, optionally, at least one surfactant;
a targeting ligand binding to a receptor expressed on the surface of retinal pigment epithelial (RPE) cells and/or endothelial cells and/or optic nerve cells; said targeting ligand being covalently coupled to said amphiphilic shell;
   for use in a method of effectively preventing or treating, in a patient, one or several of an inflammatory component, an immune-response component, an angiogenic component and a neuropathic component of a retinal disease or of an optic neuropathy.

In one embodiment, where an optic neuropathy is intended to be treated or prevented, one or several of the following components of said optic neuropathy are effectively treated or prevented: an inflammatory component, an immune-response component, and a neuropathic component of said optic neuropathy. In this embodiment, preferably, no angiogenic component is addressed, i.e. treated or prevented.

The term “endothelial cells”, as used herein, preferably and typically refers to ocular endothelial cells.

In one embodiment, effectively preventing or treating said one or several of an inflammatory component, an immune-response component, an angiogenic component and a neurodegenerative component of said retinal disease or of said optic neuropathy, manifests itself in one or several of:
an increase in intracellular availability of said drug in retinal pigment epithelial (RPE) cells and/or in optic nerve cells and/or in ocular endothelial cells;
an extended residence time of said drug in the eye, in particular in retinal pigment epithelial (RPE) cells and/or in optic nerve cells and/or in ocular endothelial cells;
an interference with the VEGF-signalling pathway in the eye;
a suppression or reduction of retinal neovascularization;
a suppression or reduction of inflammation in the eye;
a suppression or reduction of an immune-response in the eye; and
a suppression or reduction of neurodegeneration and of neuronal cell death in the eye.

In one embodiment, when there is an increase in intracellular availability of said drug in retinal pigment epithelial (RPE) cells, there may also additionally be an increase in intracellular availability of said drug in endothelial cells, e.g. those endothelial cells lining the blood vessels of the choroid and/or the retina of the eye.

In one embodiment, effectively preventing or treating said one or several of an inflammatory component, an immune-response component, an angiogenic component and a neurodegenerative component of said retinal disease or of said optic neuropathy, may also manifest itself in one or several of: a silencing of a gene, an enhancement, reduction or suppression of the homologous expression of a gene, introduction of a gene, and heterologous expression thereof, wherein, preferably, any of these manifestations occurs in retinal pigment epithelial (RPE) cells and, optionally, in endothelial cells; or preferably, any of these manifestations occurs in optic nerve cells, e.g.

In one embodiment, said patient is a mammal, preferably a human being.

In one embodiment, said retinal disease is selected from retinal dystrophy, such as hereditary retinal dystrophy; and neovascular retinal diseases, such as retinopathy of prematurity (ROP), age-related macular degeneration (AMD) and diabetic retinopathy (DR).

In one embodiment said optic neuropathy is glaucoma, in particular open-angle glaucoma or angle-closure glaucoma.

In one embodiment,
said increase in intracellular availability of said drug in retinal pigment epithelial (RPE) cells and/or in optic nerve cells and/or in ocular endothelial cells is an increase in intracellular availability of said drug in comparison to an intracellular availability of said drug in retinal pigment epithelial (RPE) cells and/or in optic nerve cells and/or in ocular endothelial cells, observed when said drug is administered as a free drug that is not comprised within a nanoparticle, wherein, preferably, said increase is an increase by a factor in the range of from 2-5; and/or
said extended residence time of said drug in the eye, in particular in retinal pigment epithelial (RPE) cells and/or in optic nerve cells and/or in ocular endothelial cells, is a residence time of said drug in the eye, in particular in retinal pigment epithelial (RPE) cells and/or in optic nerve cells and/or in ocular endothelial cells, in the range of from at least 1 day-at least 5 days; and/or
said interference with the VEGF-signalling pathway in the eye is an inhibition of the expression or activity of the VEGF-receptor, in particular of the VEGF-R2 receptor, or is an inhibition of the expression or activity of VEGF; and/or
said reduction of retinal neovascularization is a reduction of retinal neovascularization down to 50% or less, preferably down to 20% or less, more preferably down to 15% or less, of retinal neovascularization observed in an untreated retina affected by said retinal disease; and/or
said reduction of inflammation in the eye is a reduction of inflammation down to 50% or less, preferably down to 20% or less, of inflammation observed in an untreated eye affected by said retinal disease; and/or
said reduction of immune-response in the eye is a reduction of immune-response down to 50% or less, preferably down to 20% or less, of immune-response observed in an untreated eye affected by said retinal disease; and/or
   said reduction of neurodegeneration and of neuronal cell death in the eye is a reduction of neuronal cell death to 80% or more, preferably down to 50% or more, more preferably down to 30% or more, of the level of neuronal cell death observed in an untreated eye affected by said optic neuropathy.

In one embodiment, said reduction of inflammation in the eye manifests itself in a reduction of the level(s) of one or several inflammation markers, typically from an elevated level to a normal or near-normal level. A “normal” or “near-normal” level as used herein, refers to the level of such inflammation marker(s) of a patient who is not affected by an inflammation of the eye. Typical inflammation markers of relevance for the eye are CD68, Tnf-α, Ccl-2, iNos, interleukins such as I1-6, Il-1b and Egr-1.

In one embodiment, said reduction of immune-response in the eye manifests itself in the deactivation of glial cells and Muller cells as measured by a change in the level(s) of one or more of the following markers: IBA-1 and GFAP. A change in the level of GFAP can be measured quantitatively using qPCR and Western Blot and/or ELISA. The activated status of retinal microglia cells and Muller cells can be assessed using immunohistology markers for IBA-1 and GFAP.

In one embodiment, said reduction of neurodegeneration in the eye manifests itself in a decrease of retinal damage and retinal ganglion cell loss due to decreased levels of one or several of: oxidative stress, mitochondrial dysfunction, excitotoxicity, inflammatory changes, iron accumulation and protein aggregation.

In one embodiment, said receptor expressed on the surface of retinal pigment epithelial (RPE) cells and/or endothelial cells and/or optic nerve cells is selected from a G-protein coupled receptor, an integrin, and a scavenger receptor, wherein, preferably, said integrin is selected from ανβ3-integrin and ανβ5-integrin, and wherein, more preferably, said targeting ligand is selected from a peptide having an amino acid sequence RGD, a cyclic peptide having an amino acid sequence of cyclo(-Arg-Gly-Asp-D-Phe-Cys) and derivatives thereof, or wherein preferably said scavenger receptor is CD36, wherein more preferably said targeting ligand is a phospholipid.

The term “derivative”, as used herein in the context of peptidic targeting ligands, is meant to refer to peptides that retain their capability to recognize and preferentially bind to integrins, in particular ανβ3-integrin and/or ανβ5-integrin.

The term “scavenger receptor”, as used herein, is meant to refer to cell surface receptors that have the capability of binding a broad range of ligands, including but not limited to low density lipoproteins (LDLs). In particular, such scavenger receptors may have the capability to bind to a diverse range of ligands selected from lipoproteins, phospholipids, cholesterol esters, proteoglycans, carbohydrates and ferritin. A preferred example of a scavenger receptor in the context of embodiments of the present invention is CD36. In this context, a preferred targeting ligand for CD36 is a phospholipid.

It should also be noted that embodiments of the present invention encompass and envisage nanoparticles that comprise more than one targeting ligand binding to a receptor expressed on the surface of retinal pigment epithelial (RPE) cells and/or endothelial cells; for example nanoparticles in accordance with embodiments of the present invention may comprise two different targeting ligands that bind to different receptors expressed on the surface of retinal pigment epithelial (RPE) cells and/or endothelial cells and/or optic nerve cells. For example, embodiments of the present invention may comprise two different targeting ligands one of which binds to one type of receptor, and the other one of which binds to another type of receptor. As an example, if the receptors are a G-protein coupled receptor and an integrin, such as an ανβ3-integrin, one targeting ligand may bind to said G-protein coupled receptor, and the other targeting ligand may bind to the integrin. As a further example, if the receptors are two different integrins, such as an ανβ3-integrin and an ανβ5-integrin, one targeting ligand may bind to said ανβ3-integrin, and the other targeting ligand may bind to the ανβ5-integrin.

It should also be noted embodiments of the present invention encompass and envisage nanoparticles wherein a phospholipid that forms part of the amphiphilic shell may act as a targeting ligand for directing the nanoparticle(s) to a scavenger receptor expressed on the surface of cells, e.g. retinal pigment epithelial (RPE) cells and/or endothelial cells and/or optic nerve cells. In such embodiments, the phospholipid acting as a targeting ligand, may be the only targeting ligand present on said nanoparticle(s), or it may be an additional targeting ligand that is present in addition to a separate (i.e. further) targeting ligand that is specific for another receptor expressed on the surface of cells, e.g. retinal pigment epithelial (RPE) cells and/or endothelial cells and/or optic nerve cells. Without wishing to be bound by any theory or mechanistic explanation, the presence of phospholipids in/on the amphiphilic shell of the nanoparticles according to the present invention is believed to at least increase a basic affinity of the nanoparticles to cells having scavenger receptors on their surfaces. Moreover, and again without wishing to be bound by any theory or mechanistic explanation, the presence of phospholipids in/on the amphiphilic shell of the nanoparticles according to the present invention, in conjunction with an additional (separate or further) targeting ligand in/on the amphiphilic shell, is also believed to increase the specificity of binding of the nanoparticles to retinal pigment epithelial (RPE) cells and/or endothelial cells and/or optic nerve cells. An example of a useful scavenger receptor in this context is the scavenger receptor CD36. CD36 is a high affinity receptor for, inter alia, phospholipids, such as occur for example in the shell of native lipoproteins. As an example, retinal pigment epithelial (RPE) cells express CD36 on their surface and may use this to internalize natural lipoproteins, e.g. low-density lipoproteins (LDL) and very low-density lipoproteins (VLDL). The nanoparticles in accordance with embodiments of the present invention comprise phospholipids in their amphiphilic shell and are therefore able to address those receptors on the surface of RPE cells. As a consequence nanoparticle binding, uptake and enrichment in or at RPE cells may be facilitated thereby.

It should also be noted that embodiments of the present invention encompass and envisage nanoparticles that comprise more than one drug, e.g. two or three or even more drugs. The term “a core comprising a drug”, as used herein, is also meant to include a scenario where such core comprises more than one drug, i.e. it may comprise two or more drugs. For example, such scenario is also meant to encompass a scenario where such core comprises a combination of several drugs (which drugs are intended to be targeted and delivered to their preferred site of action by nanoparticles according to the present invention).

In one embodiment, said drug that has one or several of anti-inflammatory activity, immune-suppressive activity, anti-angiogenic activity, neuroprotective activity, gene therapeutic activity and regulatory activity on gene expression is selected from anti-inflammatory drugs, immunosuppressive drugs, anti-angiogenic drugs, neuroprotective drugs, nucleic acids, as well as drugs having more than one of the aforementioned qualities.

In one embodiment,
said anti-inflammatory drugs are selected from glucocorticoids, such as dexamethasone, prednisolone; COX-inhibitors, such as celecoxib, etoricoxib, rofecoxib, lumiracoxib and parecoxib; non-steroidal anti-inflammatory drugs (NSAIDs), such as acetyl salicylic acid, ibuprofen, meloxicam, diclofenac, etodolac, sulindac and indomethacin; anti-inflammatory prodrugs, such as sulfasalazine; calcineurin-inhibitors, e.g. cyclosporine A; activators of soluble guanylate cyclase (sGC), such as cinaciguat;
said immune-suppressive drugs are selected from TNF-alpha inhibitors, e.g. etanercept or adalimumab; Cyclosporins, e.g. cyclosporine A; mTOR-inhibitors, such as everolimus or sirolimus; calcineurin inhibitors, such as tacrolimus, inosinemonophosphate-dehydrogenasae inhibitors, such as mycophenolate; folic acid antagonists, such as methotrexate and methotrexate analoga; nitroimidazole-based immunesuppressants, such as azathioprine; dihydro-orotate-dehydrogenase inhibitors, such as leflunomide;
said anti-angiogenic drugs are selected from anti-VEGF-drugs, in particular inhibitors of VEGF-receptor (VEGFR) or of VEGF, such as cyclosporine A, aflibercept or ranibizumab; antifungal drugs, such as albendazole or itraconazole; folic acid antagonists, such as methotrexate and methotrexate analoga; tyrosine kinase inhibitors, such as imatinib, dasatinib, vatalanib, alectinib, sunitinib, sorafenib or erlotinib; anti-diabetics, such as glimepiride or glibenclamid; tricyclic anti-depressants, such as amitriptyline; statins, such as simvastatin, fluvastatin or atorvastatin; sartans, such as telmisartan; coumarine and coumarine derivatives; and IGF-1 receptor inhibitors;
said neuroprotective drugs are selected from immunosuppressant, anti-inflammatory and anti-oxidative drugs such as cyclosporine A (CsA) and particularly tacrolimus, Coenzyme Q10 (CoQ10), Vitamin E, citicoline (cytidine 5′-diphosphocholine), palmitoylethanolamide, melatonin, SC79 (ethyl 2-amino-6-chloro-4-(1-cyano-2-ethoxy-2-oxoethyl)-4H-chromene-3-carboxylate), Nerve growth factor (NGF), and brain-derived neurotrophic factor (BDNF); and
said nucleic acids are selected from DNA, RNA, LNA, PNA, oligonucleotides of any of the foregoing, in particular small-interfering RNA (siRNA), and microRNA (miRNA).

As used herein, the term “small interfering RNA” or “siRNA”, sometimes known as short interfering RNA or silencing RNA, refers to a class of double-stranded RNA molecules, typically 20-25 base pairs in length, that plays a role in the RNA interference (RNAi) pathway, where it interferes with the expression of specific genes with complementary nucleotide sequence. siRNA also acts in RNAi-related pathways, e.g., as an antiviral mechanism or in shaping the chromatin structure of a genome.

As used herein, the term “microRNA” or “miRNA” refers to a small non-coding RNA molecules, typically of a length of 20-500 nucleotides, more typically 20-30 nucleotides, in some instances 21-25 nucleotides, which function in transcriptional and post-transcriptional regulation of gene expression. Typically, miRNAs are encoded by eukaryotic nuclear DNA and function via base-pairing with complementary sequences within mRNA molecules, usually resulting in gene silencing via translational repression or target degradation.

In one embodiment, said nanoparticle is a lipid nanoparticle, and said shell comprises a phospholipid selected from phosphatidylcholine, phosphatidylethanolamine, phosphatidylinositol, phosphatidylserine, phosphatidic acid, phosphoinositides, phosphatidylinositol monophosphate, phosphatidylinositol bisphosphate, phosphatidylinositol triphosphate, ceramide phosphorylcholine, ceramide phosphorylethanolamine, ceramide phosphoryllipid and mixtures of any of the foregoing, and wherein said shell further comprises a surfactant, such as glyceryl ricinoleate, or lecithin, preferably a pegylated surfactant, more preferably selected from glycerol polyethylene glycol ricinoleate.

In one embodiment, said core is
a) an oily core, and said drug is a lipohilic drug, preferably selected from cyclosporine A; activators of soluble guanylate cyclase (sGC), such as cinaciguat; glucocorticoids, such as dexamethasone; statins; tacrolimus, Coenzyme Q10 (CoQ10), Vitamin E, citicoline, palmitoylethanolamide, melatonin, and SC79; or
b) an aqueous core, and said drug is a hydrophilic drug, preferably selected from anti-VEGF peptides and anti-VEGFR peptides, such as aflibercept or ranibizumab; tricyclic anti-depressants, such as amitriptyline; and growth factors such as Nerve growth factor (NGF), or brain-derived neurotrophic factor (BDNF).

In one embodiment, said core comprises an oily or aqueous phase and said drug (or said several drugs or combination of drugs), said drug being dispersed in said oily or aqueous phase, said drug preferably being dispersed in said oily or aqueous phase in the form of particles, e.g. in a nanoparticulate form, wherein, more preferably, said drug is a lipophilic drug or a hydrophilic drug, wherein, even more preferably, said drug is selected from cyclosporine A; activators of soluble guanylate cyclase (sGC), such as cinaciguat; glucocorticoids, such as dexamethasone; statins; tacrolimus, Coenzyme Q10 (CoQ10), Vitamin E, citicoline, palmitoylethanolamide, melatonin, SC79, anti-VEGF peptides and anti-VEGFR peptides, such as aflibercept or ranibizumab; tricyclic anti-depressants, such as amitriptyline; and growth factors such as nerve growth factor (NGF), or brain-derived neurotrophic factor (BDNF).

In one embodiment, said core comprises a solvent and said drug, said drug being dissolved or dispersed in said solvent, wherein, preferably, said solvent is or comprises lipids, in particular mono-, di- or trigylcerides, wherein, more preferably, the fatty acid component(s) of said mono-, di- or tri-glycerides has(have) a chain length of fatty acids in the range of from 6-18 carbon atoms, even more preferably 8-18 carbon atoms, even more preferably 8-16 carbon atoms.

In one embodiment, said nanoparticle, in particular said lipid nanoparticle, has a size, preferably a diameter, in the range of from 5 nm to 100 nm, preferably from 10 nm to 80 nm, more preferably from 20 nm to 60 nm, even more preferably from 30 nm to 60 nm.

In one embodiment, said nanoparticle, in particular said lipid nanoparticle, when administered to a patient as a sample of a plurality of nanoparticles, shows an enrichment in at least one of blood, spleen and eyes of said patient, by a factor of >3, preferably >4, in comparison to lipid nanoparticles without a targeting ligand binding to an integrin, wherein, more preferably, said enrichment is in the eyes of said patient and by a factor of >5.

The term “enrichment”, as used herein, refers to an increase in concentration of nanoparticles according to the present invention in a particular tissue or site, in comparison to nanoparticles that do not comprise a targeting ligand. Sometimes, the term “enrichment”, as used herein, is also used synonymously with “accumulation”.

The term “enrichment in the eyes” is meant to also include a scenario where such enrichment occurs in or at the optic nerve(s) (of the respective eye).

In one embodiment, said nanoparticle, in particular said lipid nanoparticle, when administered to a patient as a sample of a plurality of nanoparticles, shows an enrichment in the eyes of said patient, wherein, preferably said enrichment occurs in the retinae of said eyes or in or at the optic nerve, more preferably in the retinal pigment epithelial (RPE) cells or ocular endothelial cells or optic nerve cells, even more preferably in the retinal pigment epithelial (RPE) cells and the microvasculature of said retinae.

In one embodiment, in said method of effectively preventing or treating one or several of an inflammatory component, an immune-response component, an angiogenic component, and a neurodegenerative component of said retinal disease or of said optic neuropathy, said nanoparticle, in particular said lipid nanoparticle, is administered to a patient as a sample of a plurality of such nanoparticles, wherein such administration is performed as
a) a systemic administration, preferably selected from an intravenous administration, a subcutaneous administration, an intramuscular administration, a nasal administration, a pulmonal administration, more preferably an intravenous administration, or
b) a local administration, preferably selected from an intraocular administration, a subretinal administration, and an administration to the cornea, more preferably an intravitreal administration, even more preferably in the vicinity of the retina of the respective eye of said patient.

“Administration”, as used herein, refers to any suitable way of administering a drug. In the context of “systemic administration”, such term preferably refers to an injection, ingestion, inhalation, application or other incorporation of the drug. In a preferred embodiment, it is an injection.

In the context of “local administration”, the term refers to an injection, inhalation, application or other site-specific administration of the drug. In a preferred embodiment, it is an injection or an application.

In a further aspect, the present invention also relates to the use of a nanoparticle, in particular of a lipid nanoparticle, as defined above and herein for the manufacture of a medicament for the effective preventing or treating of one or several of an inflammatory component, an immune-response component, an angiogenic component and a neurodegenerative component of a retinal disease, e.g. of a neovascular ocular disease selected from age-related macular degeneration (AMD), diabetic retinopathy (DR) and retinopathy of prematurity; or of an optic neuropathy, in particular glaucoma, especially open angle glaucoma and angle-closure glaucoma; wherein effective treatment of said retinal disease or of said optic neuropathy manifests itself, as defined above and herein.

The present invention also relates to a method of effective prevention or treatment of one or several of an inflammatory component, an immune-response component, an angiogenic component and a neurodegenerative component of a retinal disease, e.g. of a neovascular ocular disease, as defined above, or of an optic neuropathy, as defined above, wherein said method of effective prevention or treatment comprises administering a nanoparticle, in particular a lipid nanoparticle, as defined further above and herein to a patient in need thereof.

In one embodiment, where an optic neuropathy is intended to be treated or prevented, one or several of the following components of said optic neuropathy are effectively treated or prevented: an inflammatory component, an immune-response component, and a neuropathic component of said optic neuropathy. In this embodiment, preferably, no angiogenic component is addressed.

The present inventors have surprisingly found that nanoparticles, in particular lipid nanoparticles, as defined herein are suitable to be effectively used for the effective prevention or treatment of one or several of an inflammatory component, an immune-response component, and an angiogenic component of a retinal disease, e.g. of a neovascular ocular disease, as defined herein, or neurodegenerative component of an optic neuropathy, as defined herein.

The term “inflammatory component of a retinal disease or of an optic neuropathy”, as used herein, typically refers to an inflammation associated with said retinal disease or with said optic neuropathy.

The term “immune response component of a retinal disease or of an optic neuropathy”, as used herein, typically refers to an immune response associated with said retinal disease or with said optic neuropathy.

The term “angiogenic component of a retinal disease”, as used herein, typically refers to an angiogenesis associated with said retinal disease, e.g. a neo-angiogenesis, or a proliferation of existing blood vessels, associated with said retinal disease.

The term “neurodegenerative component of a retinal disease or of an optic neuropathy”, as used herein, typically refers to neurodegenerative events and neuronal cell damage and death associated with said optic neuropathy.

The term “treatment”, as used herein, refers to an alleviation or relief or cure of one or several symptoms, and preferably the underlying pathology(pathologies), of a disease or disorder. In one embodiment, it refers to the effective alleviation or relief of a pathological inflammatory, immunogenic, angiogenic or neurodegenerative component of a disease or disorder, and to the restoration of a healthy state.

The term “prevention”, as used herein, refers to the avoidance of a pathological state occurring in a patient, or to the reduction of the extent to which a pathological state would, but for the prevention, otherwise occur in the patient.

The term “free drug” as used herein, is meant to refer to a drug that is not enclosed or compartmentalised by or within a nanoparticle, e.g. by a lipid nanoparticle, and that is, instead administered as part of a solution or dispersion or as a solid.

The term “untreated eye”, when used herein in the context of a reduction of inflammation or immune-response in the eye, refers to an eye as a point of reference that is affected by the corresponding retinal disease but that is not treated by any drug or active pharmaceutical ingredient, or at best, only by an appropriate control, such as a physiological saline solution.

The term “untreated retina”, when used herein in the context of a reduction of retinal neovascularization, refers to a retina as a point of reference that is affected by the corresponding retinal disease but that is not treated by any drug or active pharmaceutical ingredient, or at best, only by an appropriate control, such as a physiological saline solution.

The present inventors have surprisingly found that lipid nanoparticles comprising a drug can be used to effectively prevent or treat one or several of an inflammatory component, an immune-response component, an angiogenic component and a neurodegenerative component of a retinal disease or of an optic neuropathy in a patient suffering from such retinal disease, e.g. neovascular ocular disease, or suffering from such optic neuropathy.

Effective treatment of a retinal disease, such as a neovascular ocular disease, or of an optic neuropathy, manifests itself in accordance with the present invention in one or several of the following criteria:
an increase in intracellular availability of said drug in retinal pigment epithelial (RPE) cells and/or in optic nerve cells and/or in ocular endothelial cells;
an extended residence time of said drug in the eye, in particular in retinal pigment epithelial (RPE) cells and/or in optic nerve cells and/or in ocular endothelial cells;
an interference with the VEGF-signalling pathway in the eye;
a suppression or reduction of retinal neovascularization;
a suppression or reduction of inflammation in the eye;
a suppression or reduction of an immune-response in the eye; and
a suppression or reduction of neurodegeneration and neuronal cell death in the eye.

Any of the above-mentioned criteria may be an indication (or manifestation) of an effective treatment of the retinal disease, e.g. neovascular ocular disease, or of said optic neuropathy, and accordingly, such effective treatment may also be measured and/or determined by any of the above-mentioned criteria.

In one embodiment, effectively preventing or treating said one or several of an inflammatory component, an immune-response component,an angiogenic component and a neurodegenerative component of said retinal disease, or of said optic neuropathy, may also, or instead, manifest itself in one or several of: a silencing of a gene, an enhancement, reduction or suppression of the homologous expression of a gene, introduction of a gene, and heterologous expression thereof, wherein, preferably, any of these manifestations occurs in retinal pigment epithelial (RPE) cells and, optionally, in endothelial cells or retinal ganglion cells.

The term “anti-VEGF-drug” as used herein, is meant to refer to any drug that interacts with the production or action of vascular endothelial growth factor (VEGF), and/or production or action of vascular endothelial growth factor-receptor (VEGFR). Typical examples of anti-VEGF-drugs are cyclosporine A as a lipophilic drug that interferes with and suppresses the VEGF-signalling pathway. Another example is aflibercept which is a fusion protein comprising VEGF-binding portions from VEGF-receptor fused to the Fc-portion of human IgG1. Other examples of anti-VEGF-drugs are antibodies directed at VEGF or VEGFR, respectively.

The present invention also relates to a composition comprising nanoparticles, in particular lipid nanoparticles, as defined herein, which composition may then be used to effectively prevent or treat a retinal disease, such as a neovascular ocular disease, or an optic neuropathy, such as glaucoma. Typically, in such composition comprising a plurality of nanoparticles, e.g. lipid nanoparticles, as defined herein, such lipid nanoparticles are polydisperse but, preferably, with a size distribution that is narrow and, more preferably, has a polydispersity index <0.07. This allows the administration of a defined amount of drug. Because, in accordance with the present invention, the lipid nanoparticles, as defined herein, also typically have a defined encapsulation efficiency (EE) which is typically >60%, in accordance with the embodiments of the present invention, a relatively high amount of VEGF-drug may be administered in a targeted manner to its intended sight of action.

The term “nanoparticle”, as used herein, refers to a particle the average dimensions of which, in particular the size, more particularly the diameter of which, is/are in the nanometer range. Typically, such “nanoparticles” have an average diameter <500 nm, preferably <100 nm. In one embodiment, they have a size, in particular an average diameter in the range of from 5 nm to 100 nm, preferably from 10 nm to 80 nm, more preferably from 20 nm to 60 nm, even more preferably from 30 nm to 60 nm.

The term “lipid nanoparticle”, as used herein, refers to a nanoparticle that comprises one or several lipids. In one embodiment, the term refers to a nanoparticle comprising:
a core;
an amphiphilic shell, preferably a lipid shell, surrounding said core, said amphiphilic shell, preferably said lipid shell, comprising at least one phospholipid and, optionally, at least one surfactant;
a targeting ligand binding to a receptor expressed on the surface of retinal pigment epithelial (RPE) cells and/or endothelial cells and/or optic nerve cells; said targeting ligand being covalently coupled to said amphiphilic, preferably said lipid, shell.

In one embodiment, the lipid nanoparticles according to the present invention are liposomes; in another embodiment, they are lipid nanocapsules.

The term “lipid nanoparticles”, as used herein, also includes liposomes and lipid nanocapsules, as long as these have sizes in the aforementioned sense of being “nanoparticles”. Lipid nanocapsules are herein also sometimes abbreviated as LNCs. “Liposomes” are vesicles comprising one or several lipid layers, often lipid bilayers, such lipid (bi)layers acting as shell(s) and surrounding a core. Often times they are characterized by a relatively high fluidity of their shell(s) which allows such liposomes to fuse with and become part of larger particles or entities such as cells surrounded by an outer lipid membrane.

“Lipid nanocapsules”, as used herein, are nanoparticles made up of a lipid shell surrounding a core, but in comparison to “liposomes”, “lipid nanocapsules” have a higher stability and/or rigidity allowing such lipid nanocapsules to be stored for longer periods of time, e.g. for one to several months.

In one embodiment, the lipid nanoparticles according to the present invention are LDL-like. The term “LDL-like” as used herein refers to the capacity of the nanoparticles according to the present invention to act in a similar manner to low density lipoprotein (LDL) particles. Such LDL-particles have a size <60 nm, typically in the range of from 20 nm to 30 nm; they are able to diffuse through windows or junctions in endothelial cell layers, can permeate endothelial cells and are able to migrate through Bruch's membrane and into the retinal pigment epithelial (RPE) cells.

In accordance with the present invention, said lipid nanoparticles are administered as a sample/composition of plurality of nanoparticles to a patient. In one embodiment, said patient is a human patient. In one embodiment, said patient is a human patient suffering from age-related macular degeneration (AMD), diabetic retinopathy (DR) or retinopathy of prematurity (ROP), or is a human patient suffering from an optic neuropathy, in particular from glaucoma.

In accordance with the present invention, the lipid nanoparticles show an enrichment, when administered to a patient, in at least one of blood, spleen and eyes of said patient. Preferably, such enrichment is by a factor of >3, preferably >4, when compared to lipid nanoparticles which do not have a targeting ligand binding to a receptor expressed on the surface of retinal pigment epithelial (RPE) cells. In a particular preferred embodiment, such enrichment of said lipid nanoparticles is in the eyes of said patient and is an enrichment by a factor >5. In one embodiment, when such enrichment occurs in the eyes of said patient, this enrichment is preferably in the retinae of the eyes or in or at the optic nerve, more preferably in the retinal pigment epithelial (RPE) cells or ocular endothelial cells or optic nerve cells, even more preferably in the retinal pigment epithelial (RPE) cells and the microvasculature of said retinae.

For prevention or treatment of a retinal disease, e.g. of a neovascular ocular disease, or of an optic neuropathy, e. g. glaucoma, said nanoparticles, preferably said lipid nanoparticle(s) in accordance with the present invention is (are) administered to a patient as part of a sample/composition, comprising a plurality of such nanoparticles, e.g. lipid nanoparticles and at least one pharmaceutically acceptable excipient. In one embodiment, the administration is a systemic administration, preferably an intravenous injection. In another embodiment, the administration is a local administration and is, preferably, an an intraocular administration, a subretinal administration, and an administration to the cornea, more preferably an intravitreal administration, even more preferably in the vicinity of the retina of the respective eye of said patient

Abbreviations used:

LNCs=lipid nanocapsules

RGD-LNCs=lipid nanocapsules with an RGD targeting ligand.

CsA RGD-LNCs=lipid nanocapsules with an RGD targeting ligand and comrpsing cyclosporin A

CsA=cyclosporine A

Free CsA=dissolved Cyclosporin A in DPBS

DPBS=Dulbecco's Phosphate-Buffered Saline

RPE=retinal pigment epithelium

RPE cells=retinal pigment epithelial cells

MCT=medium chain triglycerides

The invention is now further described by reference to the figures wherein

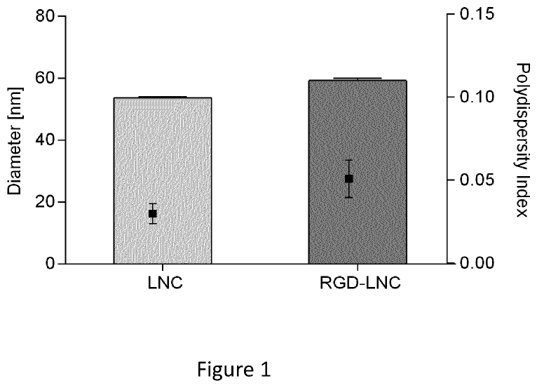

FIG. 1 shows the size and size distribution of LNCs and RGD-LNCs. Size (bars) and size distribution (polydispersity, black squares) of LNCs and RGD-LNCs were measured in 10% DPBS at 25° C. using dynamic light scattering.

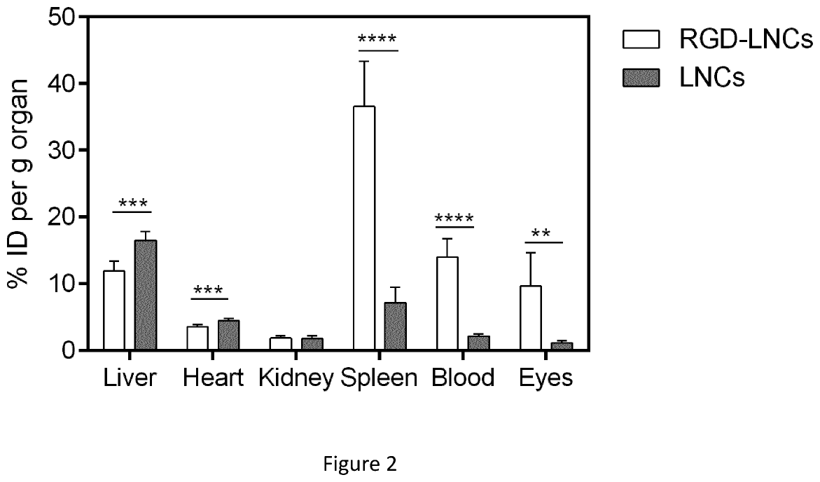

FIG. 2 shows the biodistribution of non-modified LNCs and RGD-LNCs after systemic administration and 1 h circulation time. Nanoparticle concentration in tissues was analysed by determining the fluorescence. Date is expressed as mean ±SEM (n=6). Levels of statistical significance are indicated as **(P<0.01), ***(P<0.001) and ****(P<0.0001); Results are indicated by percentage initial dose (ID) per gram organ. Percentage initial dose per gram organ after systemic administration of 100 μl of either 20 mg/ml LNCs or RGD-LNCs after 1 h circulation time.

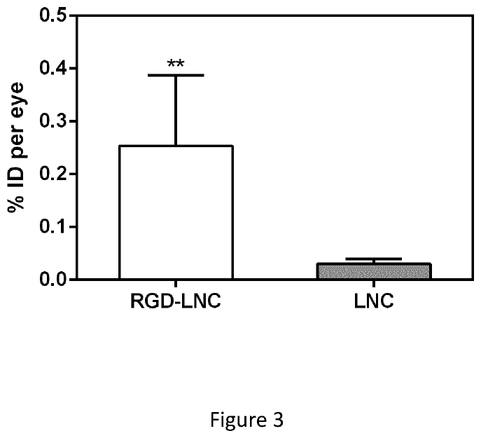

FIG. 3 shows the bioavailability of RGD-LNCs compared to LNCs after systemic administration and 1 h circulation time. Nanoparticle concentration in tissues was analysed by determining the fluorescence. Date is expressed as mean ±SEM (n=6). Levels of statistical significance are indicated as **(P<0.01), ***(P<0.001) and ****(P<0.0001); Results are indicated by percentage initial dose per eye. More specifically, FIG. 3 shows the Percentage initial dose (ID) per eye after systemic administration of 100 μl of either 20 mg/ml LNCs or RGD-LNCs after 1 h circulation time.

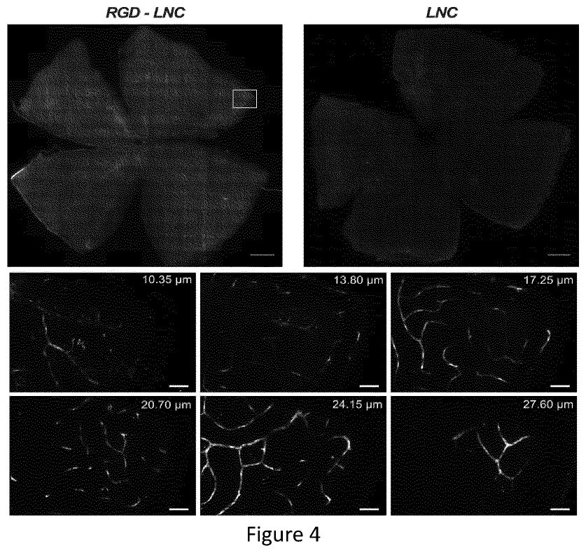

FIG. 4 shows the Microscopic analysis of flat-mounted retinas. Fluorescence images show capillary-associated nanoparticle accumulation for RGD-LNCs compared to LNCs. Z-stack pictures of RGD-LNCs treated retinas show capillary-associated fluorescence through all three retinal vascular layers (lower plexus, intermediate plexus and upper plexus). More specifically, FIG. 4 shows a Microscopic analysis of flat-mounted retinas.

TOP: Fluorescence images of whole retina show a marked increase in nanoparticle accumulation for RGD-LNCs, with a clear capillary association. Scale bar: 500 μm.

BOTTOM: In a z-stack picture of the marked area, RGD-LNC fluorescence can be clearly seen through all three retinal vascular layers. Scale bar: 50 μm.

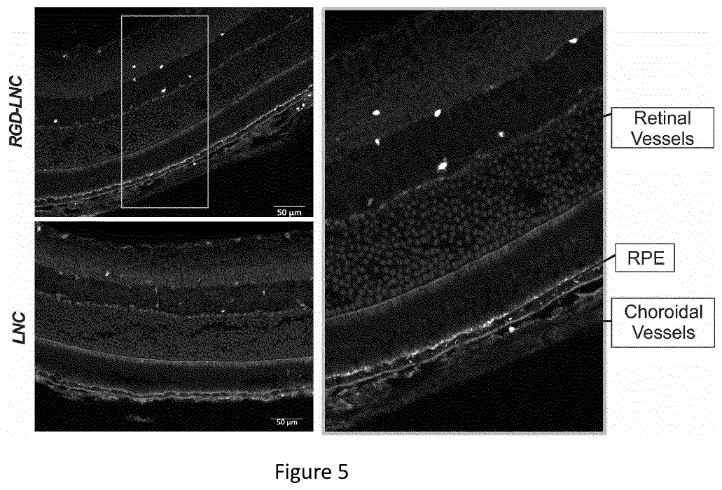

FIG. 5 shows fluorescence images of cryosection of the posterior mouse eye after the application of RGD-LNCs and LNCs (white) and 1 h circulation time, nuclei stained with DAPI (blue) and F-actin stained with Phalloidin-TRITC (orange). Revealing excessive RGD-LNC accumulation in retinal and choroidal vessels and particularly in RPE cells. More specifically, FIG. 5 shows Fluorescence images of cryosection of the posterior mouse eye after the application of RGD-LNCs and LNCs (white) and 1 h circulation time, nuclei stained with DAPI (blue) and F-actin stained with Phalloidin-TRITC (orange) for a better orientation in the tissue. Revealing RGD-LNC accumulation in retinal and choroidal vessels and particularly in RPE cells.

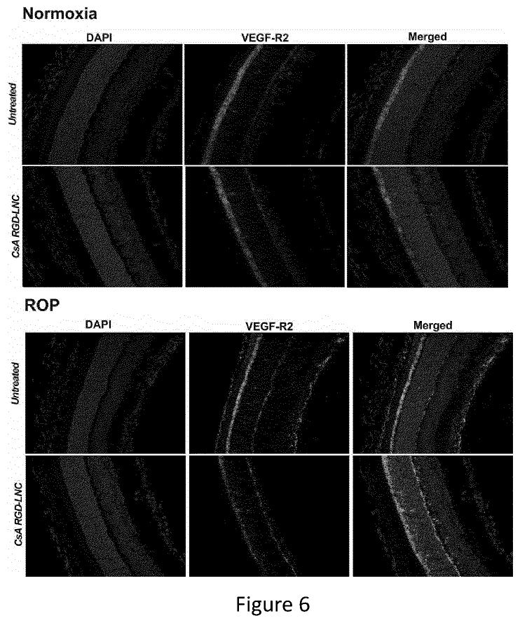

FIG. 6 shows fluorescence images of cryosections of the posterior mouse eye after the application of CsA loaded RGD-LNCs and in untreated mice. The top panel shows healthy mice (Normoxia), and the bottom panel shows mice with retinopathy (ROP). Both groups were treated at P12 with CsA RGD-LNCs or were not treated. Staining was performed using DAPI or an anti-VEGF-R2-antibody. As can be seen in the case of healthy mice, no differences between the treatment groups were observed. For mice with ROP, the treatment using CsA RGD-LNCs reduced the VEGF-R2-expression to value comparable to healthy animals.

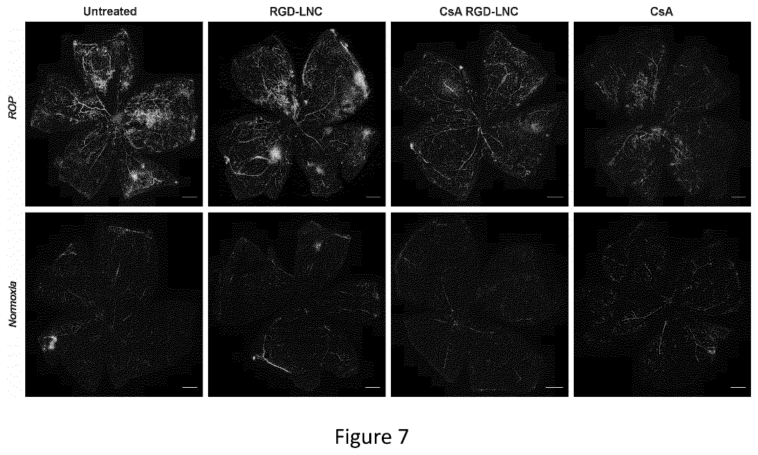

FIG. 7 shows Fluorescence images of retina whole-mounts at P17 of healthy mice (Normoxia) or mice with retinopathy (ROP), treated at P12 with either RGD-LNCs, CsA RGD-LNCs or free CsA. Revealing in the case of healthy mice, no differences between the treatment groups. For mice with ROP, the tremendous positive effect of CsA RGD-LNCs on the neovascularization can be visualized, whereas there a hardly any differences in the retinae of RGD-LNC treated and CsA treated mice compared to control. More specifically, FIG. 7 shows Fluorescence images of retina whole-mounts at P17 of healthy mice (Normoxia) or mice with retinopathy (ROP), treated at P12 with either RGD-LNCs, CsA RGD-LNCs or free CsA. Revealing in the case of healthy mice, no differences between the treatment groups. For mice with ROP, the tremendous positive effect of CsA RGD-LNCs on the neovascularization can be visualized, whereas there a hardy any differences in the retinae of RGD-LNC treated and CsA treated mice compared to control. Vessels stained with FITC-dextran. Scale bar: 500 μm.

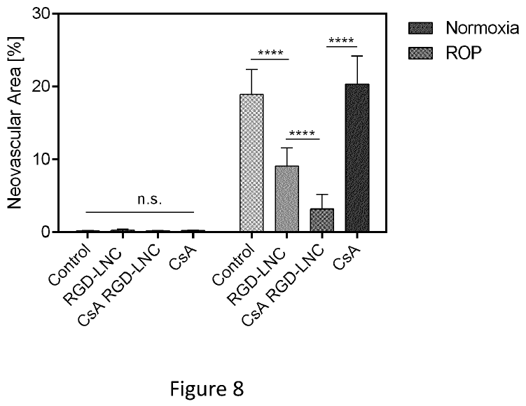

FIG. 8 shows the quantification of percentage neovascularization. Analyzation of retina whole-mounts at P17 of healthy mice (Normoxia) or mice with retinopathy (ROP), treated at P12 with either RGD-LNCs, CsA RGD-LNCs or free CsA. Revealing in the case of healthy mice, no differences between the treatment groups. For mice with ROP, the tremendous positive effect of CsA RGD-LNCs on the neovascularization can be visualized, whereas there is no effect of free CsA and a slight effect of RGD-LNCs. More specifically, FIG. 8 shows Quantification of percentage neovascularization. Analyzation of retina whole-mounts at P17 of healthy mice (normoxia) or mice with retinopathy (ROP), treated at P12 with either RGD-LNCs, CsA RGD-LNCs or free CsA. Revealing in the case of healthy mice, no differences between the treatment groups. For mice with ROP, the tremendous positive effect of CsA RGD-LNCs on the neovascularization can be visualized, whereas there is no effect of free CsA and a slight effect of RGD-LNCs.

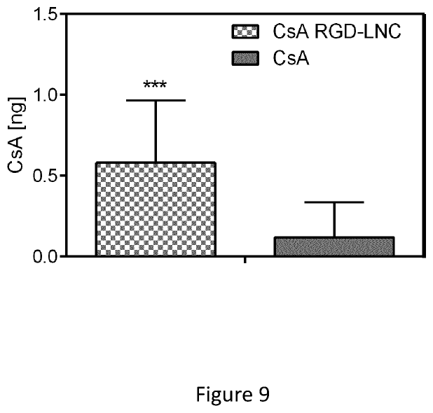

FIG. 9 shows the quantification of the total CsA amount in ng. Analyzation of eyes via UHPLC-MS at P17 of mice with retinopathy (ROP), treated at P12 with either CsA RGD-LNCs or free CsA. Revealing the presence of considerable amounts of CsA only in the eyes of mice treated with CsA RGD-LNCs, indicating the increased availability of CsA due RGD-LNC transport. More specifically, FIG. 9 shows a Quantification of the total CsA amount in the eye in ng. Analyzation of eyes via UHPLC-MS at P17 of mice with retinopathy (ROP), treated at P12 with either CsA RGD-LNCs or free CsA. Revealing the presence of considerable amounts of CsA only in the eyes of mice treated with CsA RGD-LNCs, indicating the increased availability of CsA due RGD-LNC transport.

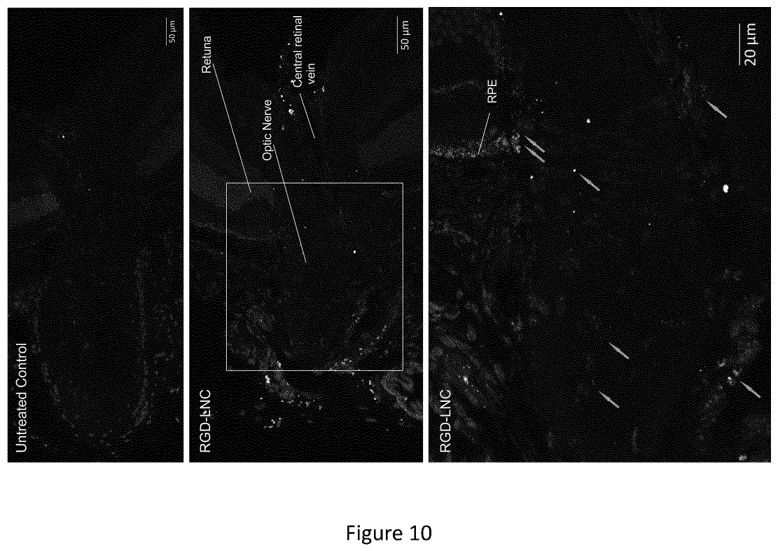

FIG. 10 shows fluorescence images of cryosection of the posterior eye of an untreated mouse or after the application of RGD-LNCs (white) and 1 h circulation time; nuclei stained with DAPI (blue). Revealing RGD-LNC accumulation in the optic nerve, especially the optic nerve head and the area of the RPE that is directly adjacent to the optic nerve.

Moreover, reference is made to the examples which are given to illustrate, not to limit the present invention.

### EXAMPLES

### Example 1

### Production of Lipid Nanoparticles

Lipid nanoparticles were chosen as a delivery system, because of their biocompatible nature, simplicity of preparation without using organic solvents and capacity to encapsulate a broad range of drugs with various solubility characteristics. Furthermore, RPE cells depend on the supply with lipids such as cholesterol, triglycerides, fatty acids and phospholipids. This requires that LDL and VLDL nanoparticles, which are classical lipid transporters, can penetrate the endothelial cell layer of the choroid and travel across the Bruch Membrane to transport lipids from the blood to RPE cells. The present inventors hypothesised that LNCs that consist of an oily core, made of medium-chain triglycerides (MCT), surrounded by a mixture of lecithin (Lipoid® S75-3) and a pegylated surfactant (Kolliphor® HS 15) would be ideal to mimic HDL and LDL. They have a thick outer layer of phospholipids similar to their biological counterparts. Their preparation was as follows:

887.5 mg Kolliphor® HS15, 30 mg Lipoid® S75-3, 415 mg MCT, 12 mg NaCl and 655.8 mg water were subjected to three cycles of progressive heating and cooling between 90 and 60° C. To quantify particles after modification and purification, fluorescent dyes (DiI, DiO or DiD 1.5% (w/w)) were added to the initial mixture. During the last cycle, an irreversible shock was induced by dilution with 5 ml water at the phase inversion temperature, leading to the formation of stable LNCs. Afterwards, additional magnetic stirring was applied for 5 min at room temperature. The final dispersion was filtered through a 0.22 μm regenerated cellulose (RC) membrane for sterilization and stored at room temperature in the dark.

To prepare drug-loaded LNCs, 35.3 mg CsA were dissolved in MCT and particles were prepared as described above.

The preparation is based on the phase-inversion temperature phenomenon of an emulsion leading to lipid nanoparticle formation with good mono-dispersity and leading to the formation of nanoparticles with a diameter of approx. 50 nm [Heurtault B. et al., A Novel Phase Inversion-Based Process for the Preparation of Lipid Nanocarriers., Pharm. Res., 19, 2002]. As a next step, the LNCs were grafted with a targeting ligand. First, cyclo(-Arg-Gly-Asp-D-Phe-Cys) (RGD) peptides were coupled to the amphiphilic DSPE-PEG2000-maleimide using conjugation chemistry between the thiol group present on the cyclic structure of the peptide and the maleimide. Next, the conjugate was inserted in the shell of the LNCs by post-insertion method, by simply heating up the mixture of DSPE-conjugate and LNCs to favour the transfer of DSPE-PEG molecules from micelles to LNCs. Modified LNCs were dialyzed against DPBS overnight using Spectra/Por® Float-A-Lyzer® G2 MWCO 300 kDa (Sigma-Aldrich, Germany) and subsequently centrifuged twice (15 min, 4000 g) using an Amicon® Ultra-4 MWCO 100 kDa centrifugal filter (Merck, Germany) for further purification.

To that end, firstly ligand molecules were coupled to the amphiphilic DSPE-PEG2000-maleimide using the conjugation chemistry between the thiol-group present on the cyclic structure of the peptide and the maleimide. Next, the conjugate or DSPE-mPEG2000 was inserted in the shell of the LNCs by post-insertion method [Perrier T., et al., Post-insertion into Lipid Nanoparticles (LNCs): From experimental aspects to mechanisms., Int. J. Pharm., 396, 2010].

As a ligand, a small, cyclic peptide was used, cyclo(-Arg-Gly-Asp-D-Phe-Cys) (RGD), that is highly potent and αvβ3 integrin specific.

### Example 2

### Targeted Administration of Lipid Nanoparticles

The targeting concept chosen by the present inventors was proved in vivo as follows: 100 μl of either 20 mg/ml LNCs or RGD-LNCs were injected systemically into healthy mice and were allowed to circulate for 1 h. Afterwards, mice were sacrificed, and the content blood, organs and eyes were collected. Blood, organs and eyes were homogenized in lysis buffer and afterwards all samples were centrifugated and nanoparticle concentration in the supernatant was measured via fluorescence. Actual Nanoparticle content was quantified using a calibration curve, made individually for each organ by spiking homogenates with defined nanoparticle amounts.

The inventors found that RGD-LNCs are able to sufficiently circulate in the blood and additionally they showed extended blood circulation in contrast to non-modified LNCs (FIG. 2). Moreover, it reveals that RGD-modified LNCs are able to accumulate efficiently in the eye, in contrast to non-modified LNCs (FIG. 3), with an enhancement of bioavailability by factor 10.

For a deeper insight in the nanoparticle localization in the eye, fluorescent microscope pictures of retina flat mounts were taken (FIG. 4). To that end, 100 μl of either 20 mg/ml LNCs or RGD-LNCs were injected systemically into healthy mice and were allowed to circulate for 1 h. Afterwards, mice were sacrificed, eyes collected and placed in 4% PFA for 1 h. Then retina was isolated and retina flatmounts were prepared. Afterwards, fluorescently labelled LNCs were detected using fluorescence microscopy. RGD-LNCs binding to the retinal microvasculature was clearly evident, whereas non-modified LNCs could hardly be found in the retina. By having a closer look into the retinal vasculature, RGD-LNCs are not only able to accumulate within all different plexus of the retina, indicated by fluorescence through the whole z-stack of the retina, but bind to smaller as well as to larger vessels (FIG. 4).

FIG. 5 shows a detailed image of the posterior mouse eye after the application of RGD-LNCs and LNCs (white) and 1 h circulation time, nuclei stained with DAPI (blue) and F-actin stained with Phalloidin-TRITC (orange) for a better orientation in the tissue. By having a closer look at the magnified section (rosa square), the accumulation of RGD-LNCs in retinal vessels as well as an additional accumulation in the RPE and the choroidal vessels can be seen. Especially compared to unmodified LNCs. These findings clearly confirm that RGD-modified LNCs are capable to target the posterior eye efficiently. Moreover, RGD-LNCs allow for double-play by targeting the site of pathologic manifestation as well as the central mainstay of pathomechanisms.

In order to take best advantage of that, an active compound that has various effects at the different locations was loaded into the nanoparticles. To that end, the drug Cyclosporin A (CsA) was chosen, as it is well known as an immunosuppressant and interferes at multiple intracellular sites with the VEGF signalling pathway [Freeman, D. J., Pharmacology and pharmacokinetics of cyclosporine., Clin. Biochemistry, 24, 1991]. CsA is able to suppress the intracellular VEGF signalling pathway and alleviates endothelial cell sprouting and proliferation. Additionally, CsA counteracts the TGFβ-related increase of VEGF production in RPE cells, the main source of VEGF in the retina [Rafiee, P., et al., Cyclosporin A differentially inhibits multiple steps in VEGF induced angiogenesis in human microvascular endothelial cells through altered intracellular signaling., Cell Comun. Sign., 2, 2004]. Furthermore, CsA possesses an anti-inflammatory potential and decreases interleukin-1β levels. In addition to that CsA restores damages of the blood-retina-barrier in an animal model of diabetes [A. Carmo, et al., Effect of cyclosporin-A on the blood-retinal barrier permeability in streptozotocin-induced diabetes, Mediators of inflammation, 9, 2000]. The fact that CsA has shown a significantly, but moderately alleviated progression of diabetic retinopathy after oral administration by transplantation patients, demonstrates the high therapeutic potential but suffer from an insufficient availability in the ocular vasculature [V. C. Chow, et al., Diabetic retinopathy after combined kidney-pancreas transplantation, Clin Transplant, 13, 1999].

Due to the high lipophilicity of CsA, it was directly dissolved in the oil phase of the initial emulsion and particles were prepared according to the standard protocol (see Example 1). High encapsulation efficiencies were achieved with values of 67% and a drug payload of 34.1 mg/g LNC.

### Example 3

### Mouse Model of Retinopathy of Prematurity

After confirming the targeting strategy in vitro and in vivo, the therapeutic concept for the treatment of neovascular ocular diseases was proven by using the mouse model of retinopathy of prematurity (ROP). This disease model is considered to be the standard model for retinopathy of prematurity and diabetic retinopathy, with the direct evaluation of neovascularization via retinal whole-mounts being the most meaningful outcome [Connor K. M., Quantification of oxygen-induced retinopathy in the mouse: a model of vessel loss, vessel regrowth and pathological angiogenesis, Nat. Protoc., 4, 2009]. Mice pups, 7 days old (postnatal day 7=P7), were exposed for 5 days to hyperoxic conditions (75±2% oxygen) in a sealed incubator. After 5 days, P12 (postnatal day 12=P12) mice were returned to room air. Maximal retinal neovascularization is known to occur 5 days after return to room air at P17 (postnatal day 17=P17). To that end, mice were treated at P12 with 20 μl of either DPBS (referred to as control), 20 mg/ml RGD-LNCs, 20 mg/ml CsA loaded RGD-LNCs or 0.68 mg/ml free CsA. After the single treatment at P12, mice were then at P17 anesthetized and perfused with 2 ml FITC dextran (green) for vessel staining. Afterwards, eyes were collected, and retina flat mounts were prepared. Those wholemounts were imaged as a whole using a fluorescence microscope. Additionally, whole retinal area and neovascular area can be measured and can be quantified as a percentage ratio.

FIG. 6 demonstrates the capacity of CsA-loaded RGD-LNCs to reduce the VEGF-R2 expression to a normal value, whereas in healthy mice, the VEGF-R2 levels are not altered. More specifically, FIG. 6 shows cryosections stained with DAPI and anti-VEGF-R2-antibody. The treatment using CsA RGD-LNCs resulted in a reduction of VEGF-R2 expression down to a healthy (“normal”) level. Indicating the interference with the VEGF-signalling pathway and the normalization rather the suppression of VEGF-R2 expression due to a endothelial and RPE cell specific anti-VEGF therapy.

Finally, the effect on retinal neovascularization was investigated. FIG. 7 depicts the visualized retinal neovascularization at P17, with detectable difference between CsA RGD-LNC treated mice and control mice, in the case of mice with retinopathy. If the mice were kept under normoxia and no retinal neovascularization can be detected, no negative impacts could be seen on retinal vessels after nanoparticle administration. Indicating that a prophylactic administration of CsA RGD-LNCs may cause no harm to the retinal vasculature. Additionally, no huge differences between control and RGD-LNC treated or CsA treated mice can be seen. The extend of neovascularization can be quantified as the percentage of neovascularization related to the whole retinal area and thereby subjective evaluation can be quantified. FIG. 8 reveals that there is no difference in the amount of neovascularization of healthy mice, independent of the treatment applied. In the case of mice with retinopathy, drug-free RGD-LNCs show a slight, but significant effect, while drug loaded RGD-LNCs seem to have a tremendous effect on the neovascularization after one-time treatment at P12 and free CsA seem to be totally ineffective. This reveals that the ligand-grafted delivery system according to the present invention is needed to achieve satisfactory results.

To prove the assumption, that a ligand-grafted delivery system is mandatory for the effectiveness of the drug, the amount of CsA at P17 of mice with retinopathy, treated on P12 was measured via UHPLC-MS. To that end, treated mice were anesthetized at P17, eyes were collected and front part of the eye, including the lens was discarded. Then posterior eye segment was placed into methanol, homogenized, centrifuged, filtered and analysed using UHPLC-MS. Samples were quantified with a calibration curve prepared from untreated mice eyes with spiked CsA. FIG. 9 reveals, that only when a delivery system is used, the there is still CsA present, indicating an extended residence time of the CsA in the eye and an increased availability of the drug.

In this model, that mimics ideally the pathomechanisms of retinopathy of prematurity, CsA loaded RGD-LNCs show tremendous effects on the overall pathogenesis, in particular on the neovascularization of the retina, as they are able to directly inhibit the neovascularization.

### Example 4

### Targeted Administration of Lipid Nanoparticles

Lipid nanoparticles, as prepared in example 2, were intravenously administered as described in example 2 to healthy mice and were allowed to circulate for 1 h. Afterwards, mice were sacrificed, the eyes were collected, fixated and cryoprotected. Afterwards, sagittal cryosections were prepared, and fluorescence microscopy images were taken upon staining with DAPI. The images are shown in FIG. 10 and demonstrate an accumulation of RGD-LNCs in optic nerve cells and the RPE area adjacent to the optic nerve thus demonstrating the possibility of effectively targeting optic nerve cells by such nanoparticles, thereby enabling an effective treatment (as opposed to a merely symptomatic treatment) of optic neuropathies, in particular of glaucoma. In accordance with embodiments of the present invention, the nanoparticles may be loaded with anti-inflammatory drugs, immune-suppressive drugs, and/or neuroprotective drugs, which may then become accumulated in the region of the optic nerve or the RPE in direct proximity of the optic nerve, due to the targeting of the nanoparticles.

The afore-mentioned examples show that the nanoparticles in accordance with the present invention make use of the novel combination of nanoparticles with a suitable ligand, such as an α_{ν}β₃-Integrin ligand (RGD), and a drug, such as Cyclosporin A. This combination enables a systemic, cell-specific therapy for all retinal diseases and optic neuropathies, in particular neovascular ocular diseases and glaucoma, which, in turn, means an improvement of the current therapeutic situation in all respects.

1-15. (canceled)

16. A method of effectively preventing or treating, in a patient, one or several of an inflammatory component, an immune-response component, an angiogenic component and a neurodegenerative component of a retinal disease or of an optic neuropathy; wherein said method comprises administering a nanoparticle to a patient in need thereof;
wherein said nanoparticle comprises:
a core comprising a drug that has one or several of the following activities: anti-inflammatory activity, immune-suppressive activity, anti-angiogenic activity, neuroprotective activity, gene therapeutic activity and regulatory activity on gene expression;
an amphiphilic shell surrounding said core, said amphiphilic shell comprising at least one phospholipid and, optionally, at least one surfactant;
a targeting ligand binding to a receptor expressed on the surface of retinal pigment epithelial (RPE) cells and/or endothelial cells and/or optic nerve cells; said targeting ligand being covalently coupled to said amphiphilic shell.

17. The method according to claim 16, wherein effectively preventing or treating said one or several of an inflammatory component, an immune-response component, an angiogenic component, and a neurodegenerative component of said retinal disease or of said optic neuropathy manifests itself in one or several of:
an increase in intracellular availability of said drug in retinal pigment epithelial (RPE) cells and/or in optic nerve cells and/or in ocular endothelial cells;
an extended residence time of said drug in retinal pigment epithelial (RPE) cells and/or in optic nerve cells and/or in ocular endothelial cells;
an interference with the VEGF-signalling pathway in the eye;
a suppression or reduction of retinal neovascularization
a suppression or reduction of inflammation in the eye;
a suppression or reduction of an immune-response in the eye; and
a suppression or reduction of neurodegeneration and of neuronal cell death in the eye.

18. The method according to claim i6, wherein said retinal disease is selected from retinal dystrophy and neovascular retinal diseases; and wherein said optic neuropathy is glaucoma.

19. The method according to claim 17, wherein
said increase in intracellular availability of said drug in retinal pigment epithelial (RPE) cells and/or in optic nerve cells and/or in ocular endothelial cells is an increase in intracellular availability of said drug in comparison to an intracellular availability of said drug in retinal pigment epithelial (RPE) cells and/or in optic nerve cells and/or in ocular endothelial cells, observed when said drug is administered as a free drug that is not comprised within a nanoparticle; and/or
said extended residence time of said drug in retinal pigment epithelial (RPE) cells and/or in optic nerve cells and/or in ocular endothelial cells, is a residence time of said drug in retinal pigment epithelial (RPE) cells and/or in optic nerve cells and/or in ocular endothelial cells, in the range of from at least 1 day-at least 5 days; and/or
said interference with the VEGF-signalling pathway in the eye is an inhibition of the expression or activity of the VEGF-receptor, or is an inhibition of the expression or activity of VEGF; and/or
said reduction of retinal neovascularization is a reduction of retinal neovascularization down to 50% or less of retinal neovascularization observed in an untreated retina affected by said retinal disease; and/or
said reduction of inflammation in the eye is a reduction of inflammation down to 50% or less of inflammation observed in an untreated eye affected by said retinal disease; and/or
said reduction of immune-response in the eye is a reduction of immune-response down to 50% or less of immune-response observed in an untreated eye affected by said retinal disease; and/or
said reduction of neurodegeneration and of neuronal cell death in the eye is a reduction of neuronal cell death down to 80% or more of the level of neuronal cell death observed in an untreated eye affected by said optic neuropathy.

20. The method according to claim 16, wherein said receptor expressed on the surface of retinal pigment epithelial (RPE) cells and/or endothelial cells and/or optic nerve cells, is selected from a G-protein coupled receptor, an integrin, and a scavenger receptor.

21. The method according to claim 16, wherein said drug that has one or several of anti-inflammatory activity, immune-suppressive activity, anti-angiogenic activity, neuroprotective activity, gene therapeutic activity and regulatory activity on gene expression is selected from anti-inflammatory drugs, immunesuppressive drugs, anti-angiogenic drugs, neuroprotective drugs, and nucleic acids.

22. The method according to claim 21, wherein
said anti-inflammatory drugs are selected from glucocorticoids; COX-inhibitors; non-steroidal anti-inflammatory drugs (NSAIDs); anti-inflammatory prodrugs,; and activators of soluble guanylate cyclase (sGC);
said immune-suppressive drugs are selected from TNF-alpha inhibitors; Cyclosporins; mTOR-inhibitors; calcineurin inhibitors; inosinemonophosphate-dehydrogenasae inhibitors; folic acid antagonists; nitroimidazole-based immunesuppressants; and dihydro-orotate-dehydrogenase inhibitors;
said anti-angiogenic drugs are selected from inhibitors of VEGF-receptor (VEGFR) or of VEGF; antifungal drugs; folic acid antagonists; tyrosine kinase inhibitors; anti-diabetics; tricycle anti-depressants; statins; sartans; coumarine and coumarine derivatives; and IGF-1 receptor inhibitors;
said neuroprotective drugs are selected from immunosuppressant, anti-inflammatory and anti-oxidative drugs; and
said nucleic acids are selected from DNA, RNA, LNA, PNA, oligonucleotides of any of the foregoing.

23. The method according to claim 16, wherein said nanoparticle is a lipid nanoparticle, and said shell comprises a phospholipid selected from phosphatidylcholine, phosphatidylethanolamine, phosphatidylinositol, phosphatidylserine, phosphatidic acid, phosphoinositides, phosphatidylinositol monophosphate, phosphatidylinositol bisphosphate, phosphatidylinositol triphosphate, ceramide phosphorylcholine, ceramide phosphorylethanolamine, ceramide phosphoryllipid and mixtures of any of the foregoing, and wherein said shell further comprises a surfactant.

24. The method according to claim 16, wherein said core is
a) an oily core, and said drug is a lipohilic drug; or
b) an aqueous core, and said drug is a hydrophilic drug.

25. The method according to claim i6, wherein said core comprises an oily or aqueous phase and said drug, said drug being dispersed in said oily or aqueous phase, said drug being dispersed in said oily or aqueous phase in the form of particles.

26. The method according to claim 16, wherein said core comprises a solvent and said drug, said drug being dissolved or dispersed in said solvent, wherein is or comprises lipids, in particular mono-, di- or trigylcerides.

27. The method according to claim 16, wherein said nanoparticle, in particular said lipid nanoparticle, has a size in the range of from 5 nm to 100 nm.

28. The method according to claim 16, wherein said nanoparticle, when administered to a patient as a sample of a plurality of nanoparticles, shows an enrichment in at least one of blood, spleen and eyes of said patient, by a factor of >3, in comparison to nanoparticles without a targeting ligand binding to an integrin.

29. The method according to claim 28, wherein said nanoparticle, when administered to a patient as a sample of a plurality of nanoparticles, shows an enrichment in the eyes of said patient, wherein said enrichment occurs in the retinae of said eyes or in or at the optic nerve.

30. The method according to claim 16, wherein, in said method of effectively preventing or treating one or several of an inflammatory component, an immune-response component, an angiogenic component, and a neurodegenerative component of said retinal disease or of said optic neuropathy, said nanoparticle is administered to a patient as a sample of a plurality of such nanoparticles, wherein such administration is performed as
a) a systemic administration selected from an intravenous administration, a subcutaneous administration, an intramuscular administration, a nasal administration, a pulmonal administration, more preferably an intravenous administration, or
b) a local administration selected from an intraocular administration, a subretinal administration, and an administration to the cornea, more preferably an intravitreal administration, even more preferably in the vicinity of the retina of the respective eye of said patient.

31. The method according to claim 18, wherein the retina disease is selected from hereditary retinal dystrophy, and the glaucoma is open-angle glaucoma or angle-closure glaucoma.

32. The method according to claim 20, wherein said integrin is selected from ανβ3-integrin and ανβ5-integrin, and wherein said targeting ligand is selected from a peptide having an amino acid sequence RGD, a cyclic peptide having an amino acid sequence of cyclo(-Arg-Gly-Asp-D-Phe-Cys) and derivatives thereof, or wherein said targeting ligand is a phospholipid.

33. The method according to claim 23, wherein said surfactant is glycerol polyethylene glycol ricinoleate.

34. The method according to claim 24, wherein said lipophic drug is selected from cyclosporine A; activators of soluble guanylate cyclase (sGC); glucocorticoids; statins; tacrolimus; Coenzyme Q10 (CoQ10); Vitamin E; citicoline; palmitoylethanolamide; melatonin; and SC79; and said hydrophilic drug is selected from anti-VEGF peptides and anti-VEGFR peptides; tricyclic anti-depressants; and growth factors.

35. The method according to claim 26, wherein the fatty acid component(s) of said mono-, di- or tri-glycerides has(have) a chain length of fatty acids in the range of from 6-18 carbon atoms.
